(19) United States
(12) Patent Application Publication
(10) Pub. No.: US 2017/0164831 A1
(43) Pub. Date: Jun. 15, 2017

(54) SYSTEM AND METHOD FOR MEASURING INTRAOCULAR PRESSURE

(71) Applicant: CALIFORNIA INSTITUTE OF TECHNOLOGY, Pasadena, CA (US)

(72) Inventors: Hyuck Choo, San Marino, CA (US); Ashwin Balakrishna, Santa Clara, CA (US); Oliver Chen, Berkeley, CA (US); Kun Ho Kim, McLean, VA (US); Jeongoen Lee, Pasadena, CA (US); Vinayak Narasimhan, Pasadena, CA (US); Haeri Park, Pasadena, CA (US)

(21) Appl. No.: 15/364,106

(22) Filed: Nov. 29, 2016

Related U.S. Application Data
(60) Provisional application No. 62/287,329, filed on Jan. 26, 2016.
(60) Provisional application No. 62/274,470, filed on Jan. 4, 2016.
(60) Provisional application No. 62/261,176, filed on Nov. 30, 2015.

(51) Int. Cl. A61B 3/16; A61B 3/00

(52) U.S. Cl. CPC A61B 3/16; A61B 3/0008; A61B 2562/0285

(57) ABSTRACT

IOP sensors and IOP measurement algorithms are disclosed herein. Examples of the IOP measurement algorithm can include a signal demodulation and artificial neural network algorithm to produce reliable results using minimal computational resources. The overall accuracy and speed of the IOP sensors and measurement algorithms enable their implementation in home-based IOP measurement systems.

### CROSS-REFERENCE TO RELATED APPLICATIONS

This filing claims the benefit of and priority to U.S. Provisional Application Ser. No. 62/261,176, filed Nov. 30, 2015, U.S. Provisional Application Ser. No. 62/274,470, filed Jan. 4, 2016, and U.S. Provisional Application Ser. No. 62/287,329, filed Jan. 26, 2016, all of which are incorporated herein by reference in their entireties for all purposes.

### FIELD OF THE INVENTION

Various aspects of the disclosure relate to intraocular pressure sensor and reader.

### BACKGROUND

Glaucoma is a leading cause of blindness, affecting an estimated four million Americans and seventy million individuals globally. As glaucoma typically affects the elderly, the aging demographic trends indicate that this disease will continue to be an ever-increasing socioeconomic burden to society. Elevated intraocular pressure (“IOP”) is a major risk factor for glaucoma, and IOP monitoring is the single most important clinical management tool.

Despite the pervasive use of IOP readings for disease monitoring and the clinically proven importance of the aggressive lowering of IOP, current clinical management is primarily based on only periodic snapshots of IOP in the doctor's office obtained every few months. The inability of patients to easily monitor their own IOPs at different times of the day or during various daily activities hinders the comprehensive understanding of the IOP profile of individual patients and the possibility of custom-tailored IOP control.

The need for better IOP monitoring in clinical ophthalmology and in disease research has been widely appreciated. Existing measurement techniques in clinical use measure IOP indirectly. Current IOP measurements involve a form of contact or noncontact application tonometry. However, both modalities have difficulties in providing reliable and repeatable readouts of actual IOP values inside the eye. All tonometers produce indirect IOP readings by deforming the ocular globe and correlating this deformation to the pressure within the eye. Their readouts are heavily influenced by the corneal curvature and thickness, or corneal mechanical properties that vary due to co-existing ocular pathologies. For example, patients who have received laser photorefractive keratectomy have thinner corneas in the treated eyes and consistently show lower IOP when measured using tonometry techniques.

Tonometry currently requires specialized equipment operated by an ophthalmologist, optometrist, or skilled technician. Hence, IOP measurements are made typically in a doctor's office about two to four times per year. Since studies show that IOP varies widely throughout the day, quarterly measurements are poor representations of a patient's actual IOP profile.

### SUMMARY OF THE INVENTION

Example embodiments of IOP sensors and IOP measurement algorithms are disclosed. Certain embodiments of the IOP measurement algorithm comprise a signal demodulation and artificial neural network algorithm to produce reliable results using minimal computational resources. The overall accuracy and speed of the disclosed IOP sensor and IOP measurement algorithm enable their implementation in home-based IOP measurement system. In this way, patients can take periodic measurements throughout the day and report irregularities to an optician. Patients may also monitor their IOP in fine time resolution throughout a physically intensive routine such as a gym work-out to better understand movements that may trigger surges in IOP levels.

Certain embodiments of the IOP sensor can include: a first wall comprising a flexible membrane; a first chamber formed by the first wall and a second wall; and a first array of photonic components disposed inside of the first chamber. In some embodiments, a raised portion is located within the first chamber on a surface of the second wall, and the first array of photonic components is disposed on a surface of the raised portion.

Further embodiments of the IOP sensor can include: a first wall comprising a flexible membrane; a first chamber formed by the first wall and a second wall; a second chamber sharing the second wall with the first chamber; a plurality of openings located on a surface of the second wall to create a plurality of pass-through openings between the first and second chambers; and a first array of photonic components disposed inside of the first chamber.

Example embodiments of methods for manufacturing an IOP sensor are disclosed. Embodiments of the method can include: providing an array of photonic components on a substrate, wherein the array of photonic components is formed by a lithography process; submitting the array of photonic components on the substrate to a thermal reflow process to transform the array of photonic components to an array of hemispherical photonic components; attaching the array of hemispherical photonic components to a surface of a chamber; and sealing a side of the chamber with a flexible membrane.

Example embodiments of an apparatus for reading intraocular pressure are also disclosed. The apparatus can include: a housing structure; a reflectance probe having a light source, the reflectance probe being attached to the housing structure; an optical spectrometer configured to receive light from an IOP sensor, the optical spectrometer being attached to the housing structure and configured to output a resonance spectrum upon receiving the light from the collimating lens; and circuitry configured to determine the intraocular pressure based on one or more variations (e.g., peaks and valleys) in the resonance spectrum.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a perspective view of an example embodiment of an IOP sensor.

FIGS. 2-4 are cross-sectional views illustrating example embodiments of IOP sensors.

FIG. 5 is a perspective view of an example embodiment of an IOP sensor.

FIGS. 6-10 are cross-sectional views illustrating example embodiments of IOP sensors.

FIGS. 11A-D illustrate an example embodiment of a method for fabricating a photonic component array.

FIGS. 12A-B illustrate example embodiments of measurements relevant to IOP determinations.

FIGS. 13 and 14 are process flow charts for signal-preprocessing, peak detection, reflectance spectra modeling and matching in accordance with example embodiments of the disclosure.

FIGS. 15A-15F are charts illustrating ANN modeling and certification in accordance with example embodiments of the disclosure.

FIGS. 16A-16G are charts illustrating pressure, amplitude, and frequency profiles produced by an example embodiment of the ANN SDA based algorithm.

FIGS. 17A-17B illustrate example optical readout apparatuses in accordance with example embodiments of the disclosure.

FIG. 18 is a block diagram illustrating an example of a hardware implementation for an apparatus employing a processing system that may exploit the systems and methods of FIGS. 1-17B in accordance with some embodiments of the disclosure.

### DETAILED DESCRIPTION

### Overview

As previously mentioned, glaucoma is one of the most prevalent and perplexing disease today. By 2020, it is projected that 79.6 million people worldwide will have the disease. Of those 79.6 million, 4.5 million of them will suffer from irreversible bilateral vision loss. Currently, IOP is the most modifiable risk factor for glaucoma and moderating elevated IOP levels by individualized medication or surgery is the only available therapeutic modality. IOP has a circadian rhythm that fluctuates between 10-21 mmHg throughout the day. This makes it difficult to diagnose and suggest treatments to patients based on sparse IOP measurements taken at a clinic. For example, in one study, a peak IOP measurement from a continuous 24 hour-monitoring-period was on average 5 mmHg higher than a peak IOP measurement taken at the clinic. This false low pressure reading at the clinic may result in improper treatment in 80% of the patients. As such there is a need for a fast and accurate IOP measurement sensor and method that could be implemented in a home-based IOP measurement system.

Current clinic-based IOP measurement technology such as Dynamic Contour Tonometry (DCT) is not suitable for a home-based implementation because it is both expensive and bulky. DCT is capable of sampling 100 IOP values per second with ±1 mmHg sensitivity. However, it requires very high computer processing power, which is one of the reasons why DCT is expensive and bulky (large computer equipment). Other existing IOP measurement technologies such as LC sensor implants, micro-fluidic channel sensors, and strain gauges lack the sensitivity and sampling time resolution compared to DCT. For example, LC sensor implants have a sensitivity of 2.5 mmHg and micro-fluidic channel sensors have a sensitivity of 0.5 mmHg but lacks the sampling speed and rate. Accordingly, there is a need for fast and accurate an IOP measurement technology that could be implemented in both commercial and home-based IOP measurement systems.

Generally, home-based IOP measurement technologies seek to achieve two performance goals: (1) high sensitivity: to measure IOP in sub-1 mmHg scale; and (2) high sampling frequency: to detect acute IOP fluctuations by obtaining an IOP profile with high temporal resolution. Provided herein are example embodiments of IOP sensors and IOP measurement methods that are both highly sensitive and faster than conventional techniques. These embodiments can have a sensitivity of ±0.01 mmHg and microsecond level processing time per signal. This is a considerable improvement in both accuracy and speed over existing IOP measurement technologies (e.g., DCT, LC sensor, micro-fluidic channel, and strain gauges).

In certain embodiments disclosed herein, the sensor for sensing biological pressure (e.g., intraocular pressure) can include an implantable device comprising a first membrane structure, an optional second membrane structure, and a plurality of photonic components adapted to reflect light, wherein the first and second membrane structures are separated by a gap and the first membrane structure is movable with respect to the second membrane structure in response to a change in ambient (surrounding) pressure such that the device has a resonance frequency that shifts as a size of the gap changes. A detection device can be adapted to transmit optical light to the implantable device and detect the resonance frequency of the implantable device based on at least one wavelength of light reflected from the implantable device. The detection device can be adapted to detect the resonance frequency based on a magnitude variation of the at least one wavelength of light reflected from the implantable device. The detection device can be adapted to determine the biological pressure based on the detected resonance frequency of the implantable device.

In some embodiments both the first and second membrane structures are deformable in response to the change in ambient pressure. Both the first and second membrane structures can be separated by one or more mechanical flexures. Also, both the first and second membrane structures can be rigid in some embodiments.

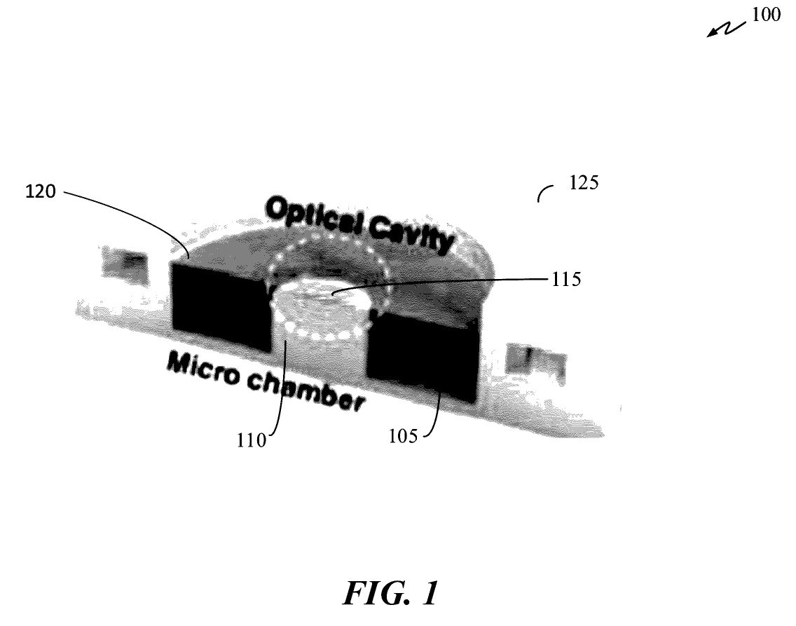

FIG. 1 illustrates an example embodiment of an IOP sensor 100 in accordance with aspects of the disclosure. IOP sensor 100 is a light-driven intraocular pressure sensing implant with a high sensitivity of ±0.01 mmHg. IOP sensor 100 can be placed between the cornea and the iris in the patient's eye. To determine the intraocular pressure, IOP sensor 100 can be excited by an excitation beam (infrared light with wavelength between 700-1150 nm) from an external light source of a portable reader unit. The reflected light from the IOP sensor 100 can then be processed by an optical spectrometer located in the portal reader unit. The optical spectrometer is configured to determine a resonance spectrum from the reflected light.

The resonance spectrum can then be processed by an IOP measurement algorithm that first converts the resonance spectrum in real time with a signal demodulation algorithm (SDA). The IOP measurement algorithm further processes the demodulated signal using an artificial neural networks (ANN) based algorithm. This combination of SDA and ANN algorithms produces IOP readouts with ±0.01 mmHg sensitivity and microsecond level processing time per signal. The SDA-ANN algorithm can represent a vast improvement in accuracy and speed over conventional technologies, which enables IOP sensor 110 and the SDA-ANN based algorithm to be implemented in home-based IOP measurement systems. For example, a commercially available DCT system (with a conventional IOP measurement algorithm) is capable of sampling 100 IOP values per second, but requires high computer processing power. The disclosed SDA-ANN based algorithm has a microsecond level processing time per signal (faster than current DCT system) without the need for an expensive and high-processing-power computer system.

### IOP Sensor

Referring again to FIG. 1, IOP sensor 100 includes a hermetically sealed cavity or chamber 105, a raised portion 110, an array of photonic components 115, a flexible membrane 120, and a protective layer 125. In some embodiments, chamber 105 may have a circular shape and may be further defined by a raised portion 110 at the center of chamber 105. Raised portion 110 may create a donut-shaped chamber. As shown in FIG. 1, raised portion 110 is shaped like a cylinder or has a circular cross-section. However, it should be noted that the cross-section of raised portion 110 may have other shape such as a square, a rectangle, an oval, etc. Chamber 105 may also exhibit other shape such as a square, a rectangle, a triangle, or an oval. Additionally, raised portion 110 may be centrally disposed at the center of chamber 105.

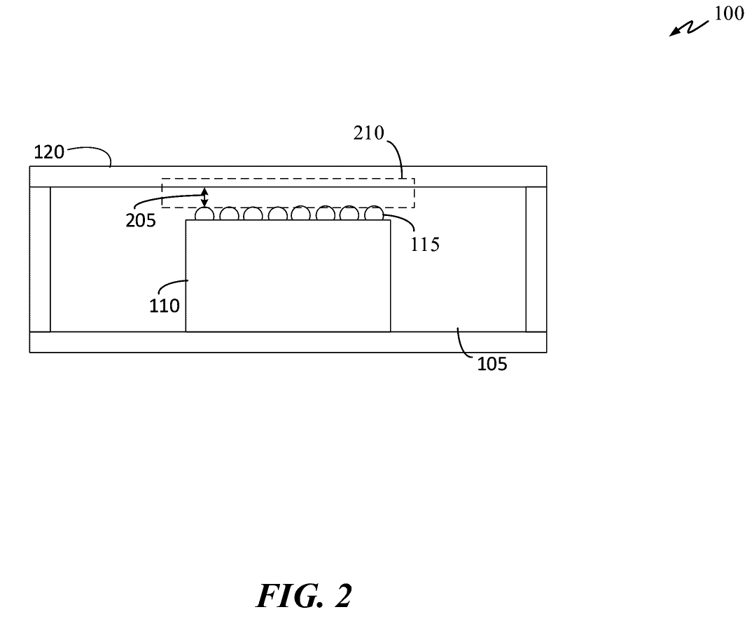

The array of photonic components 115 may be disposed on a surface of raised portion 110. The photonic components described herein are components of the sensor and broadly serve to facilitate the reflection of light (photons) by the sensor. The photonic components, as will be described herein, can have various sizes and shapes, and can be arranged in arrays or patterns of various designs. FIG. 2 illustrates a side view of IOP sensor 100 as shown in FIG. 1. IOP sensor 100 will now be described with reference to both FIGS. 1 and 2. In some embodiments, each nanodot of the array of nanodots 115 has a diameter between 200-300 nm. In some embodiments, the diameter of each nanodot is 240 nm. Nanodot array 115 may have an overall dimension of 50 μm by 50 μm, and the distance between the center of each nanodot may be between 250-350 nm. In some embodiment, the distance between the center of each nanodot may be 300 nm. The above noted diameter, overall dimension, and the distance between the center of each nanodot are example dimensions of some embodiments and are no way inclusive as other dimensions may be used.

Each photonic component may be cylindrical in shape. In some embodiments, each photonic component has a hemispherical shape, which has a wide angle of reflectance thereby enabling light to enter at a wide range angle of incidence. In other words, the light source on the external reader does not have to be directly shone (zero angle of incidence) at IOP sensor 100 (which can be embedded in the eye of a patient). One benefit to the hemispherical shape across the array is a smoother reflectivity distribution of the optical resonance spectrum as sharper transition edges (between each photonic component) lead to a deeper dip in the reflectivity distribution. This also contributes to a wider-band resonance spectrum and less sensitivity to the angle of incidence of the light source. In some cases, the photonic components can be referred to as nanodots, but this term does not require the components to be sized on a “nanoscale” nor have a round shape absent explicit recitation of such in the claims.

In some embodiments, cavity 105 may include an optical cavity 210, which can be formed by a distance 205 between a horizontal tangential plane of nanodot array 115 and the bottom surface of flexible membrane 120. Distance 205 changes as flexible membrane 115 deforms toward or away from nanodot array 115 due to the changes in the intraocular pressure. Distance 205 determines the distribution of the reflectance spectra. In turn, one or more variations (such as a peak or valley) of the resonance spectrum can be used to determine the intraocular pressure. In some embodiments, distance 205 has an initial distance range of 5-10 μm. In some embodiments, distance 205 has an initial range of 7.3 μm.

As shown in FIGS. 1 and 2, flexible membrane 120 forms one of the walls of chamber 105. In some embodiments, flexible membrane 120 may have a thickness between 0.2 to 0.5 μm. In another aspect, flexible membrane 120 has a thickness of 0.34 μm. At this thickness, the membrane's flexibility is optimum and the interference factor with infrared light is minimum. In some embodiments, flexible membrane 115 may include of a silicon-nitrite material such as SiN or Si₃N₄. Other transparent and flexible materials may also be used. However, at a minimum, the flexible material must not interfere with infrared light having wavelengths between 700 nm-1150 nm. The use of infrared light having wavelengths between 700 nm-1150 nm can be desirable because light at this range of wavelength is not detectable by the human eye as visible light may cause the patient to blink and/or squint. Additionally, near infrared light has excellent tissue penetration and its reflectance can be easily detected by a sensor.

Because flexible membrane 115 can be very thin and potentially fragile, protective layer 125 may be disposed along the outer perimeter of membrane 115 but not to cover any portion of chamber 105. Protective layer 125 can be designed to give surgeons a surface to grab onto IOP sensor 110. This allows for the installation of IOP sensor 110 into a patient's eye without touching flexible membrane 115. In some embodiments, protective layer 125 may be made of silicone.

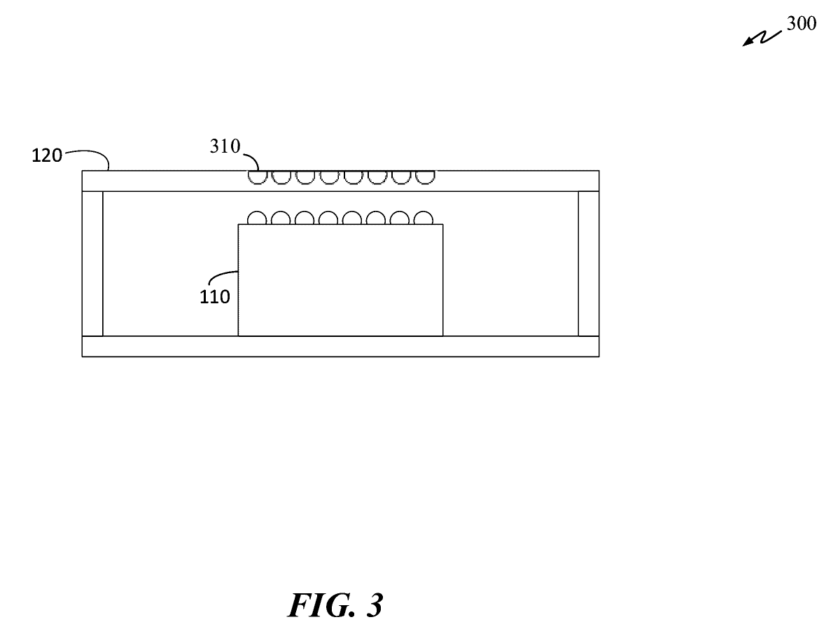

FIG. 3 illustrates an IOP sensor 300 in accordance with certain embodiments of the disclosure. IOP sensor 300 includes all of the features of IOP sensor 100 as previously described but with the addition of a second array of nanodots 310 embedded within flexible membrane 120. In some embodiments, nanodot array 310 may be hemispherical or fully spherical. Nanodot array 310 may be embedded near the surface of membrane 120 away from raised portion 110. Alternatively, nanodot array 310 may be embedded near the surface of flexible membrane 115 that faces chamber 105.

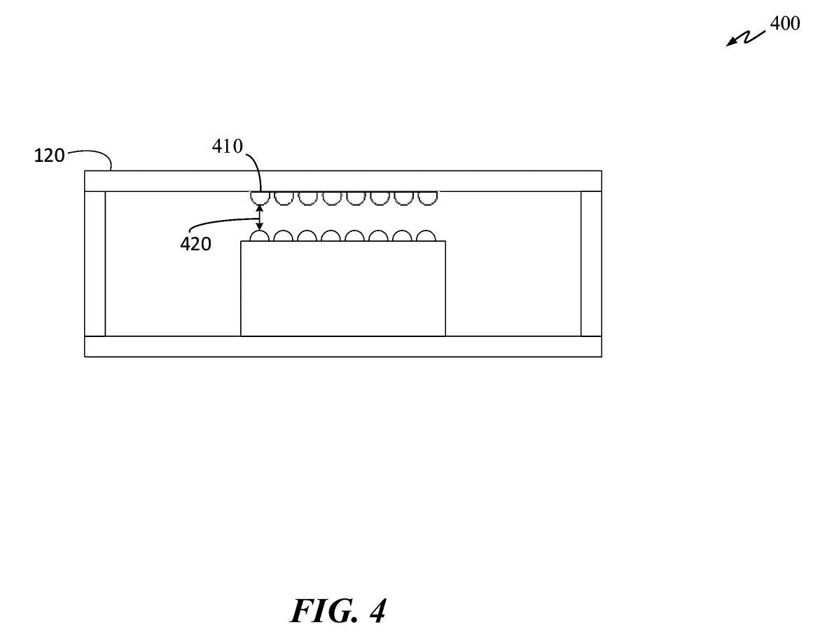

FIG. 4 illustrates an IOP sensor 400 with a second array of nanodots 410 being located on an inner surface of flexible membrane 115. The inner surface can be the surface facing chamber 105. In IOP sensor 400, the diameter of each nanodots in each of the arrays may be configured to achieve an optimum optical distance 420.

### 2-Chamber IOP Sensor

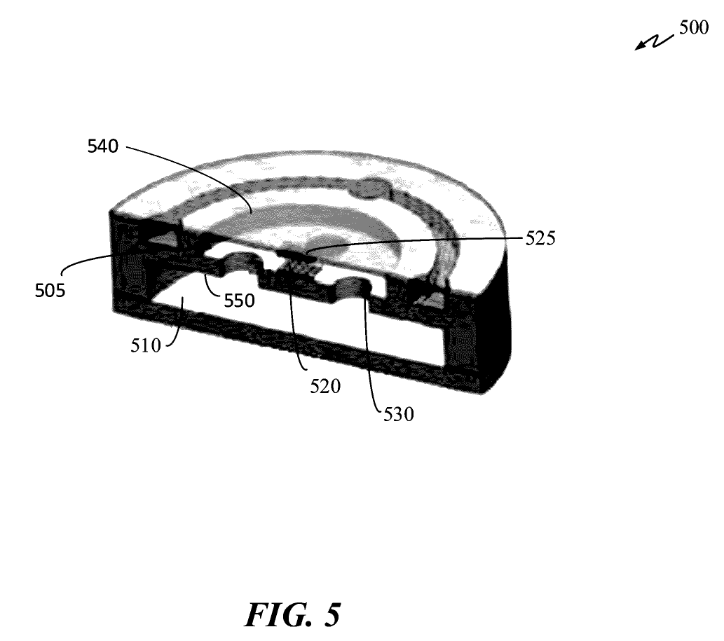

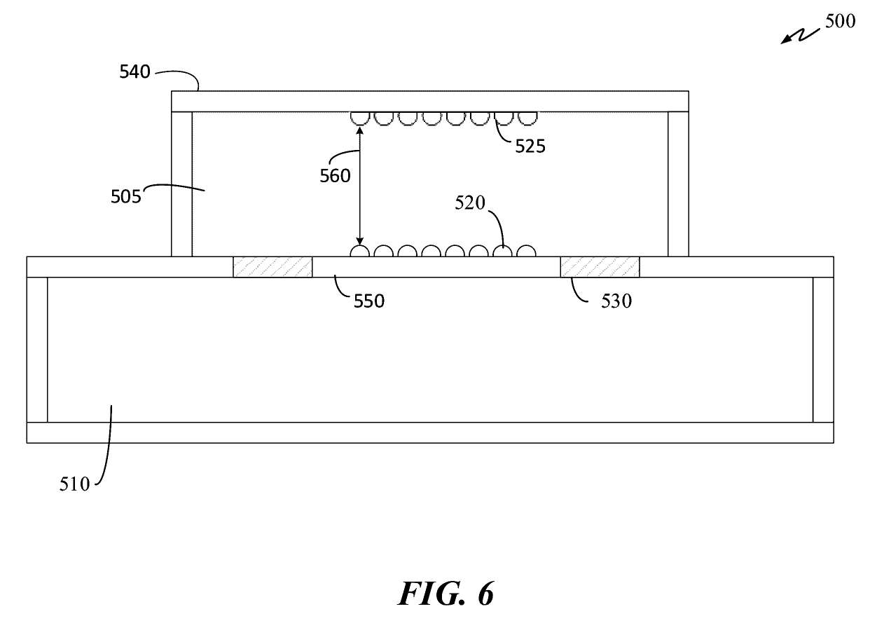

FIG. 5 illustrates a perspective view of an example embodiment of a two-chamber IOP sensor 500 in accordance with aspects of the present disclosure. FIG. 6 illustrates a cross-sectional view of the same IOP sensor 500. IOP sensor 500 includes a first chamber 505, a second chamber 510, a first array of nanodots 520, a second array of nanodots 525, a plurality of holes 530, and a flexible membrane 540. In some embodiments, chambers 505 and 510 are vacuum sealed. Alternatively, chambers 505 and 510 are filled with an inert gas at a pressure between 10-21 mmHg, which is the normal range of pressure inside the eye.

In some embodiments, the first array of nanodots 520 can be disposed on a surface of a wall 550, which forms one of the two walls of chamber 505. Wall 550 can be the middle wall, which forms chamber 505 and chamber 510. Flexible membrane 540 forms the second wall of chamber 505. As shown, the first array of nanodots 520 may be disposed in the center of surface 550. Although not shown, the first array of nanodots may be disposed on a raised surface, which raises the first nanodot array 520 toward flexible membrane 540.

The second array of nanodots 525 may be disposed at a bottom surface (e.g., the surface closer to chamber 505) of flexible membrane 540. The space between the first and second nanodot arrays forms an optical cavity. The optical cavity may have a thickness 560 of approximately 7.4 microns. It should be noted that other suitable thickness may be employed.

As shown in FIGS. 5 and 6, IOP sensor 500 has a second chamber 510. Chambers 505 and 510 are separated by wall or layer 550. In some embodiments, layer 550 includes a plurality of holes 530 evenly distributed around nanodot array 520. Layer 550 may have 2 or more holes 530. In some embodiments, layer 550 may have 4 holes 530. Each of the holes 530 can be the same size. In some embodiments, holes 530 are circular holes. Alternatively, holes 530 may have other shape such as a square, a rectangle, a triangle, an oval, etc. In some embodiments, second chamber 510 is larger in size than chamber 505. Alternatively, both chambers 505 and 510 may have the same size.

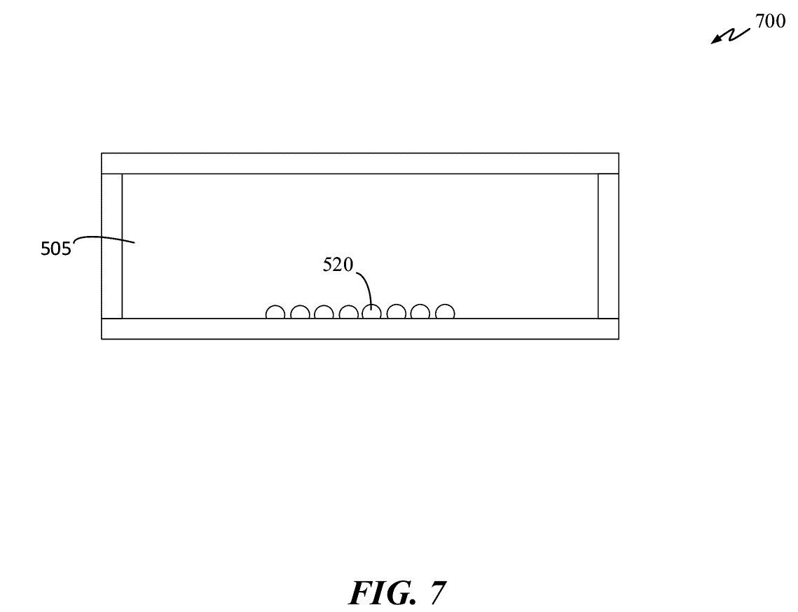

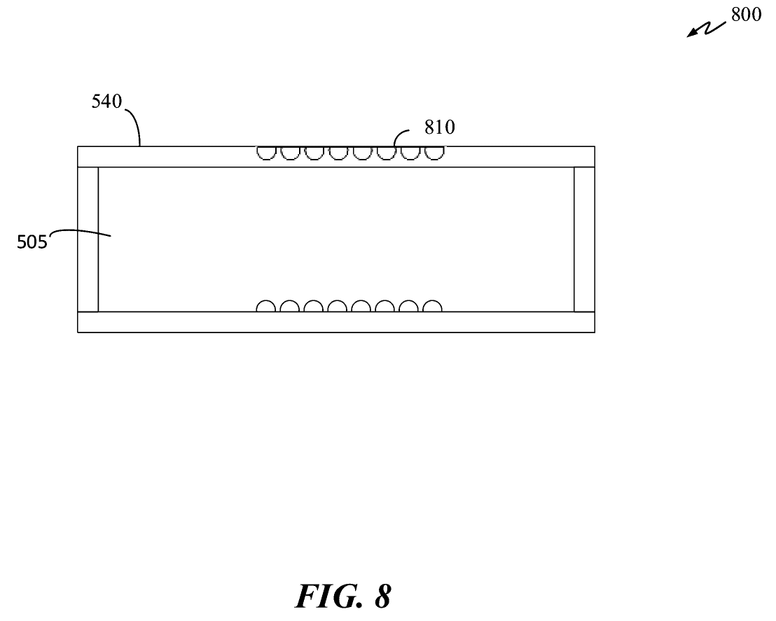

FIG. 7 illustrates an example embodiment of an IOP sensor 700 in accordance to aspects of the disclosure. IOP sensor 700 can be similar to IOP sensor 500 but with only one array of nanodots 520 in chamber 505. FIG. 8 illustrates an example embodiment of IOP sensor 800, which is similar to IOP sensor 500 but the second array of nanodots 810 can be embedded within flexible membrane 540 instead of at an inner surface of flexible membrane 540.

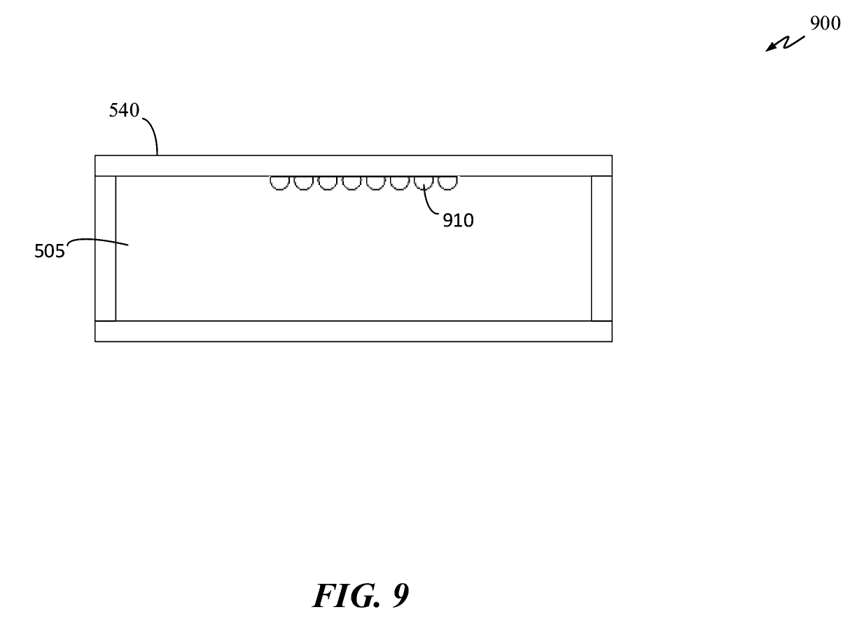

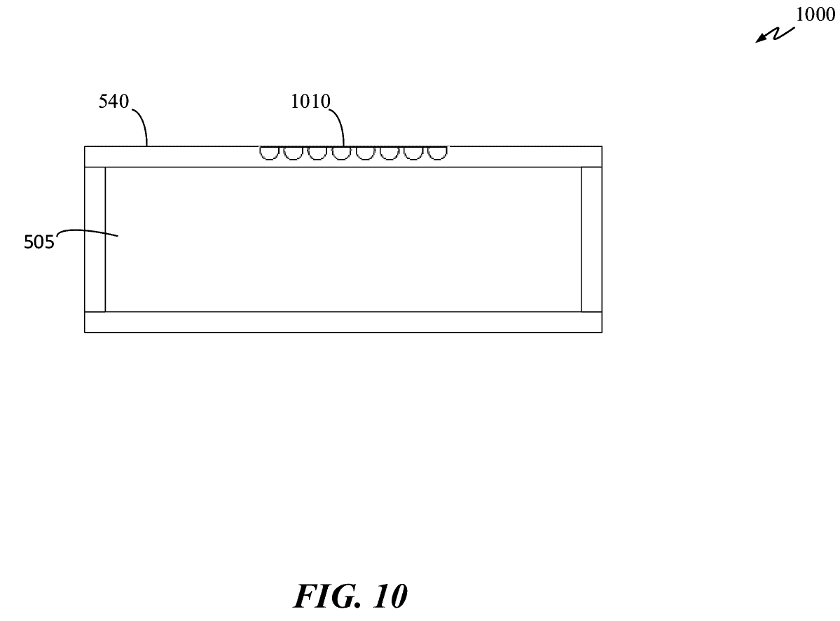

FIG. 9 illustrates an example embodiment of an IOP sensor 900 in accordance to aspects of the disclosure. IOP sensor 900 can be similar to IOP sensor 500, but with only one array of nanodots 910 being located at the inner surface of flexible membrane 540. FIG. 10 illustrates an example embodiment of an IOP sensor 1000 in accordance with aspects of the disclosure. IOP sensor 1000 can be similar to IOP sensor 900, but with an array of nanodots 1010 being embedded within flexible membrane 540. It should be noted that each of nanodot arrays 310, 410, 520, 525, 810, 910, and 1010 may include one or more features of nanodot array 115 as previously described. For example, each of the nanodot arrays may have a nanodot diameter of 240 nm and the distance between the center of each of the nanodot in an array may be 300 nm. Each nanodot may be cylindrical, hemispherical, or fully spherical in shape. Additionally, each of the array may have a dimension of 50 microns by 50 microns. Other dimension may also be used as long as a sufficient amount of reflective light is received by the optical spectrometer.

### Nanodot Array Fabrication

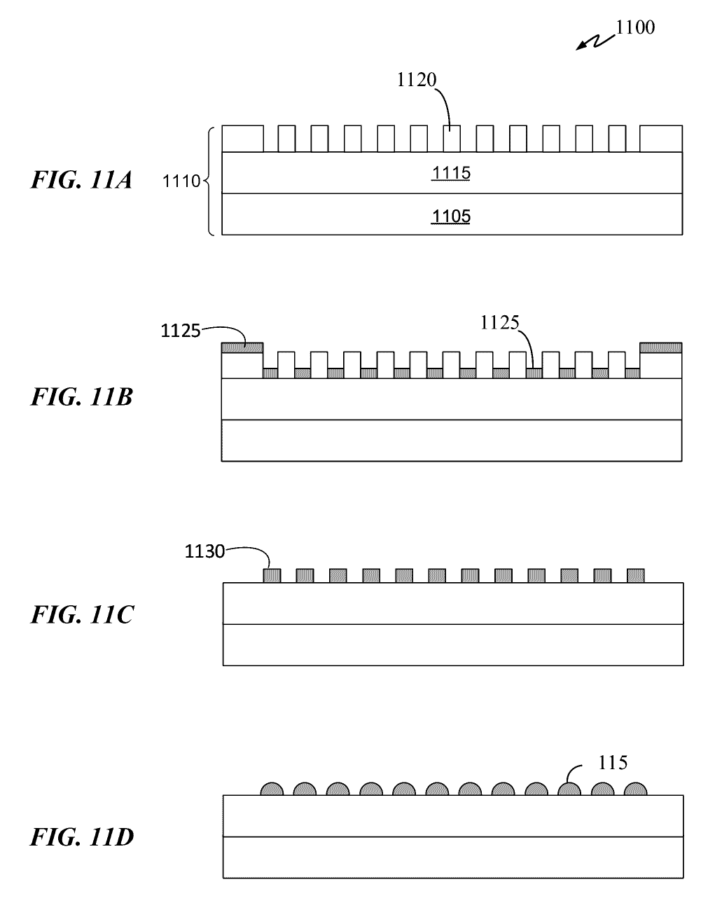

FIGS. 11A-D illustrate an example embodiment of a process 1100 for fabricating nanodot array 115 in accordance with aspects of the disclosure. In FIG. 11A, a substrate 1110 having a preformed pattern of nanodot array 1120 is provided. Substrate 1110 can be fabricated by first providing a base substrate 1105 and then coating base substrate 1105 with a layer of flexible material 1110. In some embodiments, layer 1110 may be coated onto substrate 1105 using evaporation or physical vapor deposition (PVD) such as sputter deposition (which may include reactive sputtering, ion-beam sputtering, or ion-assisted sputtering).

Next, a layer of photoresist 1120 can be coated onto layer 1115. Photoresist layer 1120 is then exposed and developed to form an array of nanodots. In general, there are two types of photoresist: a negative and a positive acting resists. A negative photoresist goes through a photo-hardening process when exposed to ultraviolet (UV) light. A positive photoresist goes through a photo-softening process during UV exposure and leaves the same pattern on the resist film after UV exposure. It should be noted that the substrate 1110 may be produced using both positive or negative resist. In some embodiments, a positive resist can be used to ultimately create the nanodot array pattern on photoresist layer 1120. Once photoresist layer 1120 is patterned and exposed to UV light (cured), it goes through a chemical bath to lift off the portion that is exposed to the UV light. This leaves behind a pattern of holes where the nanodots will be formed.

If a negative resist is used, then a negative pattern of nanodots will be patterned onto substrate 1115. Patterning may be done using photolithography, which employs a mask. Alternatively, the array of nanodots pattern can be directly patterned onto substrate 1115 using electron beam lithography.

As depicted in FIG. 11B, a gold layer 1125 can be deposited using PVD or other suitable coating technique. As depicted in FIG. 11C, the remaining photoresist layer 1120 may be removing using a lift off or stripping process. What remains after the stripping process is an array of nanodots 1130 before being subjected to a thermal reflow process. It should be noted that other types of metal may also be used such as platinum or copper.

As depicted in FIG. 11D, the array of nanodots can be subjected to a thermal reflow process. During the reflow process, the temperature is raised to the melting point of the metal (gold). Once the metal/gold nanodots are cooled, they take a spherical shape and form nanodot array 115. As previously mentioned, each nanodot of the array of nanodots 115 has a diameter of 240 nm. Additionally, nanodot array 115 may have an overall dimension of 50 μm by 50 μm, and the distance between the center of each nanodot may be 300 nm.

### IOP Measurement Algorithm

To obtain IOP measurements, an illuminant probe can be configured to shine near infrared (NIR) light at IOP sensor 100. The NIR light may have a wavelength between 700-1150 nm. In this range, the NIR light is invisible to the human eye. This range of wavelength can be desirable because it is invisible to the human eye. Further, this range of wavelength has an excellent tissue penetration property. Because IOP sensor 100 may incorporate hemispherical nanodot array, the illuminant probe does not have to shine the NIR light directly at sensor 100. The hemispherical shaped nanodot array enables the array to receive light at a wider range of angle of incidence and still can reflect light back at a near perpendicular angle with respect to the horizontal plane of the nanodot array.

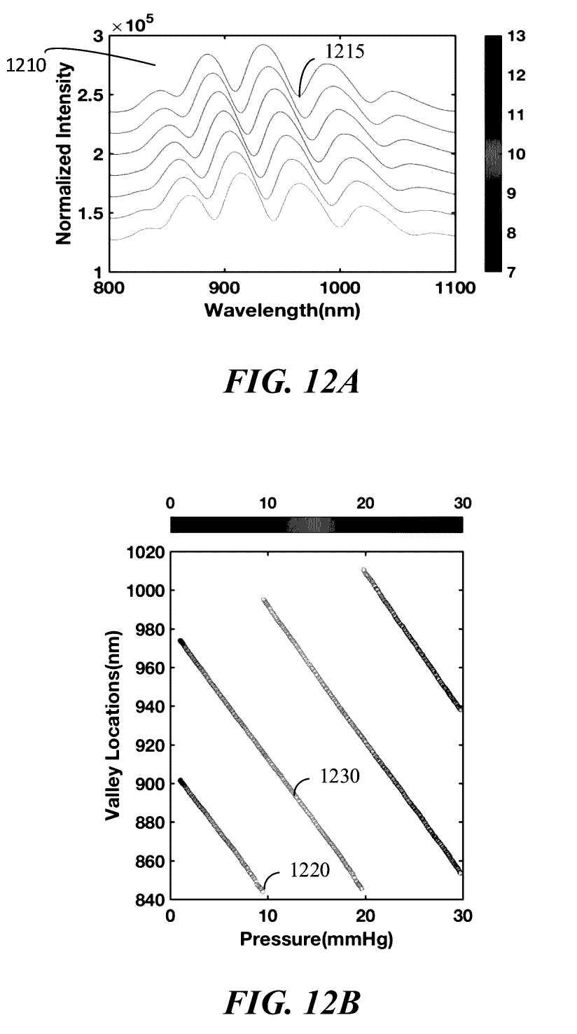

The reflected light (also referred to as a reflection spectrum) is then captured by an optical spectrometer that is running the SDA-ANN algorithm. In some embodiments, the SDA-ANN enabled optical spectrometer receives the reflected spectrum and produces a resonance waveform 1210 as shown in FIG. 12A. Referring to FIG. 12A, a blue shift in the spectrum occurs when there is an increase in ambient pressure. While a decrease in the ambient pressure causes a red shift in the spectrum. In some embodiments, the location of the resonant spectrum minima (e.g., valley 1215) provides information for signal demodulation as it is uniquely determined by the extent of the deflection of flexible membrane 120 (shown in FIGS. 1 and 2).

FIG. 12B is an optical spectrum shift chart that illustrates the non-linear relationship between valley locations and the intraocular pressure. As shown in FIG. 12B, the valley locations are initially linearly correlated with the ambient pressure within the 0˜10 mmHg pressure range. However, the left most valley 1220 vanishes around 10 mmHg and the right adjacent valley 1230 becomes the new left most valley in the 10˜20 mmHg range. Because of the jump from one valley line to another (vanishing valley effect) as the ambient pressure moves the full ambient pressure range (e.g., 0 to 30 mmHg), the relationship between valley location and pressure becomes non-linear. This presents a challenging problem for demodulation—transformation from the Euclidian space to the pressure space. The SDA-ANN algorithm disclosed herein helps resolve this non-linearity issue. In some embodiments, an ANN regression can be used to demodulate the resonance waveform. Once the waveform is demodulated, an accurate reading of the intraocular pressure may be obtained.

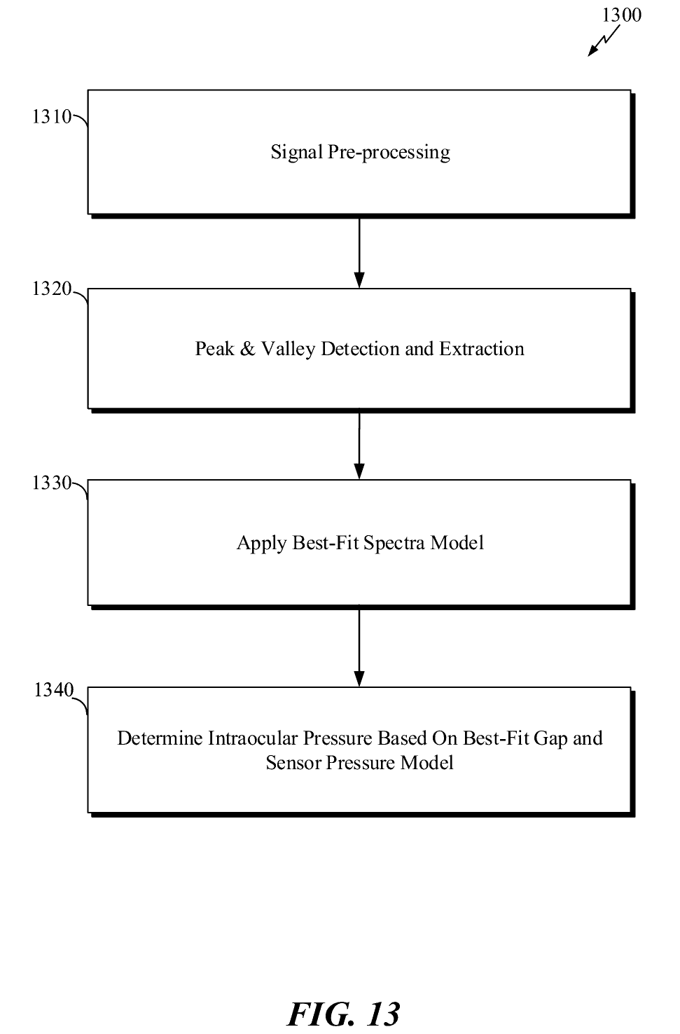

FIG. 13 illustrates an example embodiment of an IOP measurement process 1300. IOP measurement process 1300 includes a signal demodulation and an artificial neural network algorithms. The SDA is part of signal pre-processing stages 1310 and 1320. ANN is part of processing stage 1330 as shown in FIG. 13.

At stage 1310, denoising and low pass filtering can be performed on the signal. This preprocessing step can be performed to identify and compensate for misalignments between the illuminant probe and IOP sensor 100. The first step of stage 1310 can be to categorize the misalignment between the illuminant probe and the IOP sensor using a valley detection module. By appropriately categorizing the misalignment, the valley detection module can instruct the user to make the appropriate correction in real-time in order to achieve a better or optimum signal. Generally, there are two orientation conditions—transverse and longitudinal—that should be met in order to obtain proper resonance spectra. First, the light probe should be fixated at a transverse coordinate within a valid focal range in order to prevent over and under-reflection. Second, the convergence point of the light should be in close proximity to the sensor diaphragm center in order to mitigate peripheral reflections from the cavity contour. When these three positioning conditions are not satisfied, the reflection spectrum becomes saturated, which will overwhelm optical sensors in the spectrometer. Alternatively, incorrect probe positioning may also lead to no signal or cause the reflection spectra to have a single peak waveform resulting from the black-body radiation profile of the light source.

In some embodiments, when a saturated signal is detected, the valley detection module may generate an instruction to instruct the user to adjust the focal length of the illumination light source to be shorter. In other words, a saturated signal indicates that the focal point the illumination light source is too long and extends beyond optical cavity of IOP sensor 100. Alternatively, when no signal is detected, the valley detection module may generate an instruction to direct the user to adjust the focus length and make it shorter. A no signal indicates that the focal length of the light source is too short. Additionally, when a single peak spectra occurs, an instruction can be generated to direct the user to translate the probe in the horizontal direction with respect to the eye or IOP sensor 100. In some embodiments, the instruction generated to the user may be audio, textual, graphical, or a combination thereof.

In some embodiments, the valley detection module can be modified to recognize misalignment waveforms such as saturation, single peak intensity, or no signal. For example, minima detection may be converted into a peak detection problem by inverting the spectrum. In some embodiments, a minimum peak prominence threshold may be set to detect both the peak intensity and low prominence peak (no signal), both of which indicate a misalignment. Further, the gradient of the intensity spectrum may be swept to check for extended regions having zero slope to filter out misalignment caused by saturation. In some embodiments, the peak detection function (of the valley detection module) may output the location of the valleys that met the threshold requirements. If no valleys were detected, the optical detector and IOP sensor are misaligned. In this case, the main program terminates with an IOP value of zero and the valley detection module informs the user of a misalignment in the set up. If more than one valley was detected, the algorithm proceeds to input the extracted valleys to the trained ANN.

In some embodiments, the illumination probe may be coupled to one or more servomotors or linear actuators and IOP process 1300 may be configured to send instruction to a controller to automatically adjust the illumination probe based on the SDA alignment analysis. In this embodiment, the illumination probe and optical spectrometer may be mounted on a stationary reader such as a portable table-top unit.

As mentioned, part of the misalignment detection algorithm includes spectral feature recognition algorithm that detects valleys. At stage 1320, on a high level, if the valleys are found to meet the detection thresholds, the SDA may conclude that the setup is well-aligned and proceeds to the ANN execution step.

In some embodiments, the first step of the valley detection module can be to smoothen the raw intensity silhouette(v_1) in order to counteract white noise from impairing the accuracy of valley detection. Next, a finite impulse response filter can be employed to compute the moving average of the 20 consecutive data points to attenuate the high frequency noise components. The additive inverse of the intensity spectrum can be then inputted into a peak detection module in order to convert the problem of minima detection into maxima detection, and thereby allowing the usage of powerful peak detection functions included in software packages such as MATLAB.

In some embodiments, three thresholds were integrated into the peak detection in order to track the desired valley locations. A minimum peak prominence (p_min) threshold was set in order to filter out misalignment (ii) and (iii) both of which display a single low prominence peak. Second a minimum peak distance(d_min) threshold was applied to deter remnant noise in the vicinity of one peak from being detected as multiple peaks. Lastly, the gradient of the intensity spectrum was swept to check for extended regions having zero gradients to filter out misalignment (i).

In some embodiments, the signal demodulation algorithm at stage 1320 includes a peak detection function configured to output the location and prominence of the valleys found satisfying the requirements set by the thresholds, which can be the minimum peak prominence threshold. If no valleys were detected, the probe and IOP sensor are misaligned. In this case, the main program module outputs the discovered misalignment with an IOP value of zero. If more than one valley was detected, the algorithm proceeds to input the extracted valleys to the ANN stage (stages 1330 and 1340) where the best theoretical spectra for each measured spectrum is best fitted. Once this is done, an air gap and sensor pressure relationship may be accurately obtained based on the best-fit spectra to pressure model.

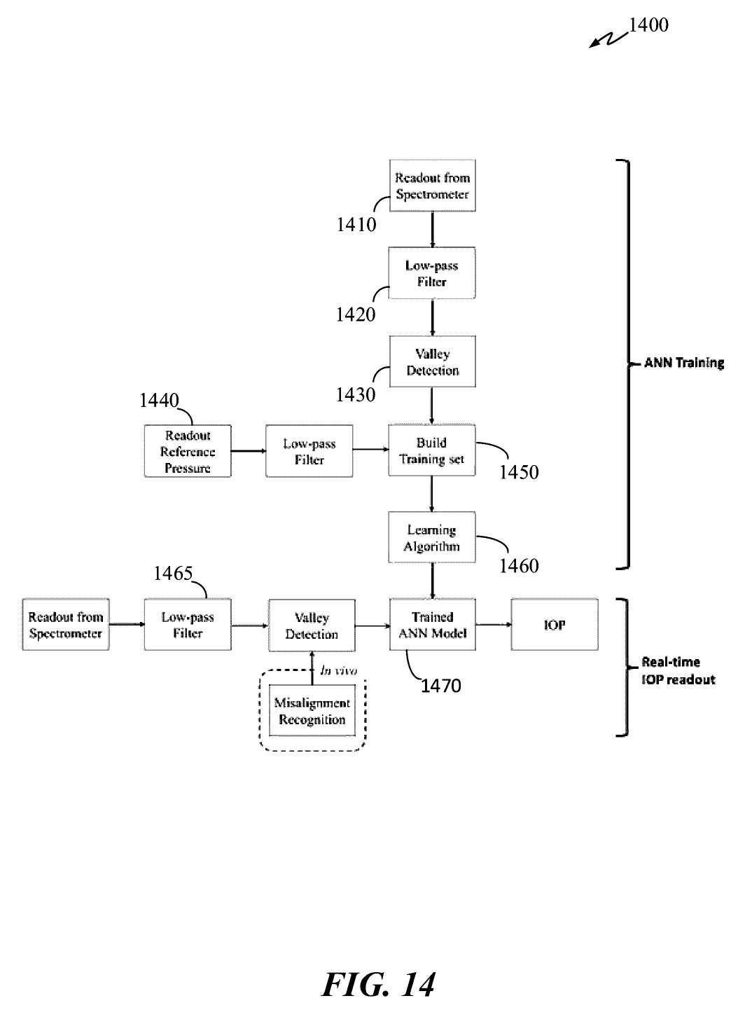

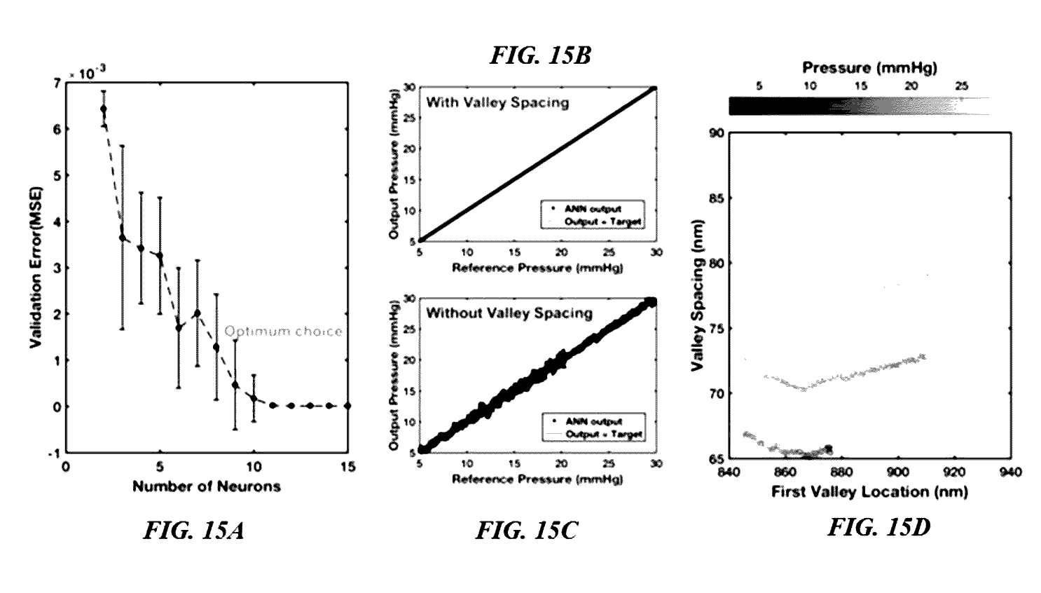

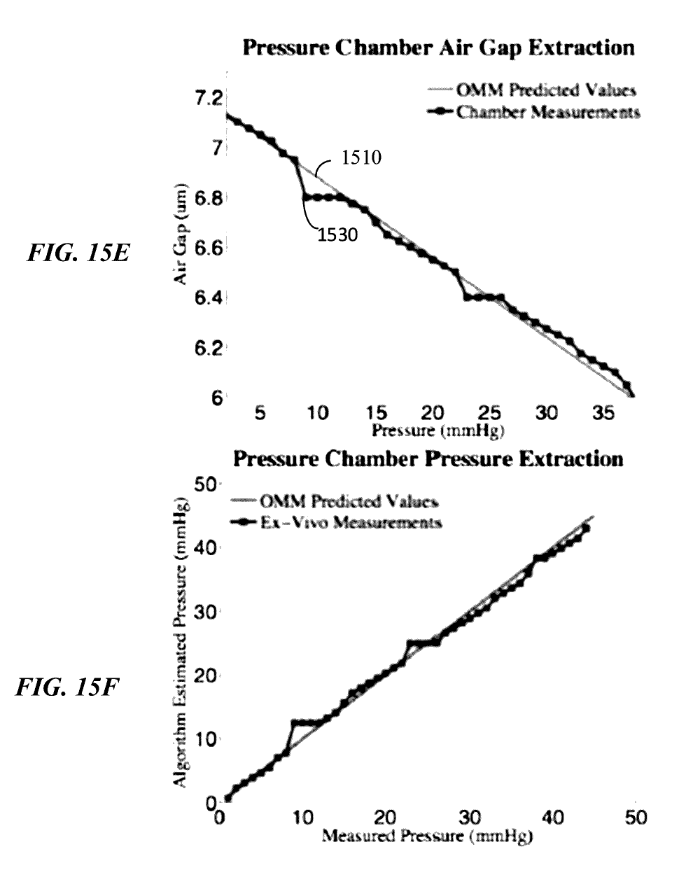

In some embodiments, the ANN algorithm includes a hidden layer with 10 neurons activated by a hyperbolic tangent non-linearity and one linear output layer. The mean squared error was used as the loss function for training. A deeper architecture was not necessary and could potentially lead to overfitting since one hidden layer was sufficient to achieve excellent out-of-sample performance. In some embodiments, the optimum number of neurons was found by cross validation in which the dependence of the estimated test accuracy on the number of hidden units was evaluated. The results are shown in FIG. 15a, which shows that the mean cross validation accuracy seizes to improve significantly after 10 neurons. In one embodiment, the ANN algorithm includes a hidden layer with more than 10 neurons (such as 11 neurons).

In some embodiments, a training procedure may be implemented to train the ANN algorithm prior to using the ANN algorithm to measure a real-time IOP from a patient's eye. FIG. 14 illustrates how a training procedure 1400 can be implemented to train the ANN algorithm in accordance to aspects of the disclosure. Each training set can be constructed by extracting the valleys (at 1430) from a raw spectrometer readout (at 1410) and labeling each set of valleys with the corresponding pressure value obtained from the reference pressure sensor (1440). All signals are passed through a low-pass filter (at 1420a and 1465) in order to attenuate high-frequency electrical noise. After the ANN is trained with a learning algorithm (1470), it can be applied for real-time spectrum demodulation. During the implementation stage, the same valley detection scheme can be applied to the spectrometer readout and the extracted valleys are input into the trained ANN model which outputs the real-time IOP.

In some embodiments, a feature vector can be used to train the ANN algorithm. The feature vector can be represented as an array as xᵢₙₚᵤₜ=[λ₁, λ₂, λ₃,Δλ₁₂, Δλ₂₃], which includes a fixed number of valleys and the wavelength difference between each adjacent pair. These valley spacing features are then weighted based on the corresponding pressures in the training set, which will be used to predict an output intraocular pressure based on the features of any arbitrary optical spectrum. Referring back to FIG. 12B, since the valley vanishing point can be arbitrary, the ANN algorithm can be configured to detect valleys having a hard minimum wavelength threshold (λₘᵢₙ) greater than λₘᵢₙ=855 nm. In some embodiments, the valley spacing was included as a feature in order to obtain higher accuracy.

In training the ANN algorithm, the network can be trained with and without the valley spacing parameter and a great improvement in calibration accuracy can be achieved when spacing was included. FIG. 15B shows the validation set accuracy with the spacing parameter included while FIG. 15C shows the validation set accuracy without the valley spacing parameter. The ANN algorithm trained without the valley spacing feature had 3 orders of magnitude higher out-of-sample mean-squared error of 1.27×10⁻² mmHg while with the spacing the error was on average 6.24×10⁻² mHg. Accordingly, it was concluded that spacing can be an important parameter that aids the learning algorithm in finding an optimal solution. In FIG. 15D illustrates the left most valley and its distance from the right adjacent valley with the corresponding IOP labels. As shown in FIG. 15D, it can be inferred that a legitimate feature vector has been chosen since a true target function exists that uniquely maps each feature vector to an intraocular pressure.

In some embodiments, the ANN algorithm has unit training set of 40000 points recorded during a linear rise in pressure. Multiple rounds of unit training sets were generated and summed up into one training set and fed into the ANN learning algorithm. A training session took on average 2.5 seconds and 550 epochs. The loss function with respect to the training epochs is shown in FIG. 15A trained ANN took on average 124.58 microseconds to process one spectrum. Likewise, an advantage of using ANNs can be the minimal amount of computation time and resources required to implement a trained model. A trained ANN can easily be implemented with miniaturized computational devices that have lower processing power which can be an important attribute in terms of moving towards a more compact readout system. The low processing time of our SDA also enables IOP process 1300 to obtain a high-temporal resolution IOP profile which has not demonstrated by existing IOP measurement technologies.

In some embodiments, an optomechanical model can be used to determine the intraocular pressure in IOP sensor 100. The optomechanical model consists of two steps. First, a model was developed to determine the air gap between the sensor's silicon nitride membrane and its silicon base given an IOP (see line 1510 in FIG. 15E). Then, a standard Fabry-Perot model was used to generate a theoretical reflectivity spectrum based on the air gap values (see 1530). To validate the mechanical model used, the air gap profile was modeled in COMSOL and a close match was found between the analytical large deflection model (see FIG. 15F).

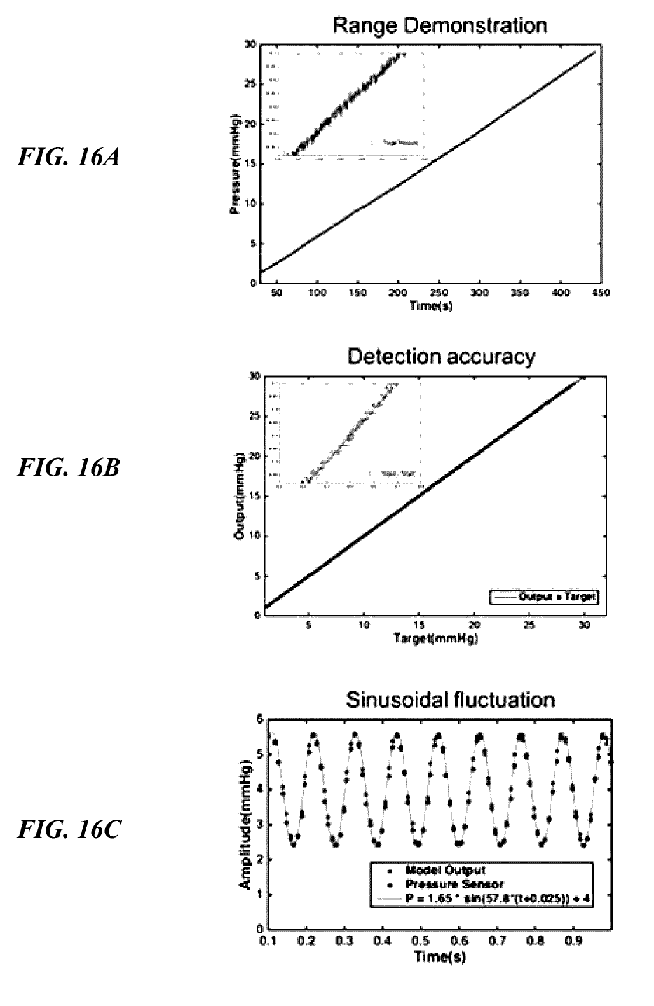

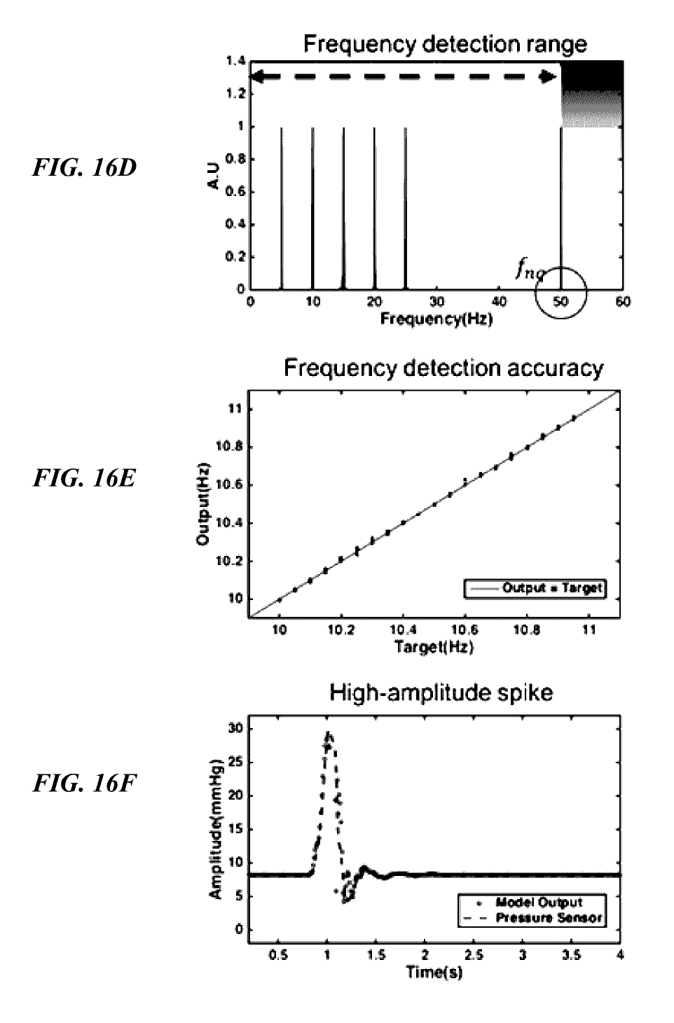

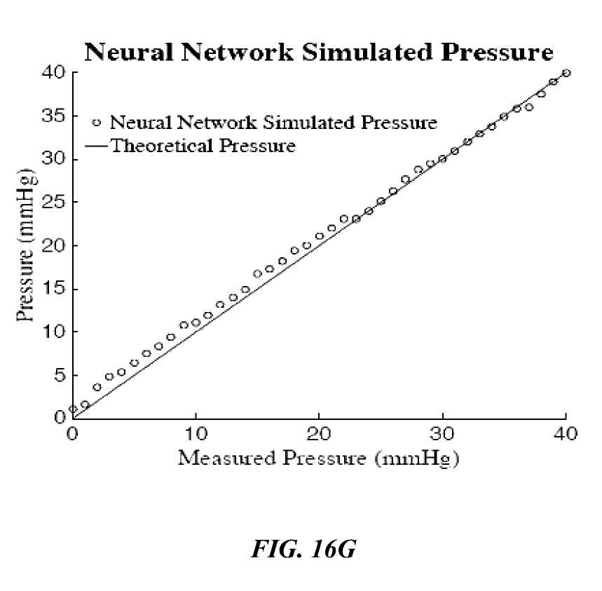

In some embodiments, the ANN algorithm was trained to perform within a clinically relevant 0 to 30 mmHg range which corresponds to the pressure range of interest. To test the neural network calibration accuracy in the full detection range, the pressure response of the IOP sensor can be compared with the readings from the reference pressure sensor for three pressure cycles, each of which is from 1 mmHg to 30 mmHg (see FIG. 16A). In some embodiments, the mean squared error between the ANN function output and the reference pressure sensor output was 6.24×10⁻⁵ mmHg. Moreover, by performing a correlation analysis on the ANN output and the reference pressure sensor values we determined the correlation coefficient (1−R=2.1457e−6) and the p-value (p≈0), both of which advocated excellent linearity as demonstrated in FIG. 16B. Moreover, the repeatability of IOP sensor 110 is 0.043% FS (0.0131 mmHg) without any observed hysteresis. Considering these infinitesimal error statistics, the ANN algorithm (of IOP process 1300) can be capable of achieving at least an equivalent pressure readout accuracy as the reference pressure sensor.

IOP process 1300 has high temporal resolution which enables it to track transient IOP spikes. To appreciate the sampling frequency of the ANN based SDA of IOP process 1300, the response of the ocular implant to a high frequency pressure fluctuation may be analyzed. FIG. 16C displays the output pressure of the IOP sensor vs the reference pressure sensor in response to an oscillatory pressure fluctuation applied at a driving frequency of approximately 9 Hz. In some embodiments, a sinusoidal curve of the form y=A sin(ωt÷φ)+B with a preset value of ω=2πf_{applied}=2π(9.2)≈57.8 Hz was fitted to the experimental data to assess the accuracy of the ANN output waveform. (A=1.65 mmHg, φ=1.445, and B=4). At the driving frequency of approximately 9 Hz, a close match of the SDA output with the applied sinusoidal pressure gradient can be observed.

FIG. 16D illustrates the range of the frequency tracking capabilities of IOP process 1300. The frequency detection has a range between 0˜50 Hz. This is shown by plotting the Fast Fourier Transformation (FFT) plot of the time-series pressure data. As shown in FIG. 16D, singular well-defined peaks at each of the frequencies applied to the system up to 50 Hz can be observed. At 50+Hz, one Fourier resonance peak at the applied frequency along with another peak mirrored at about 50 Hz was identified. This cutoff phenomenon with the theoretical frequency detection upper bound of the system is at the Nyquist frequency (or folding frequency), which is determined to be half of the sampling rate. Since the sampling rate was around 10 milliseconds, the Nyquist frequency of our detection system was fₛ/2=50 Hz which matches the experimental observations. Note that the spectrometer integration time of 9 ms is the limiting factor slowing down the overall IOP extraction time per spectrum. With the use of a higher-tier spectrometer with a minimum integration time of 100 μs below, this system has the capacity to detect frequencies up to 2500 Hz assuming the overall spectrum acquisition time can be reduced to the execution time of the ANN which was around 124 μs. As most meaningful IOP fluctuations which are pointers to a patient's health will not exceed the range of 50 Hz, here we will go as far as to demonstrate the frequency detection capabilities using a miniature spectrometer with mediocre performance.

The accuracy of the frequency detection can be demonstrated by examining the FFT spectrum while varying the applied frequency in 0.05 Hz increments from 10 Hz to 10.95 Hz. The resulting output frequency vs target frequency is illustrated in FIG. 16E. The mean squared error between the output and target frequencies was 5.2504×10⁻⁶ Hz and a linear correlation test yielded R=0.997 and p=8.61e−155. These results show that the ANN achieves high accuracy in the frequency domain.

The frequency detection experiments described above was conducted with medium amplitude (≈4 mmHg) fluctuations in pressure. To verify the aptitude of the system in detecting acute high-amplitude fluctuations of pressure, a 20 mmHg pressure spike was induced using a syringe pump and compared the results from the reference pressure sensor with the ANN output. The erratic pulsatile waveform plotted in FIG. 16F demonstrates a close agreement between the reference sensor readout and the ANN output pressure validating the ability of the detection system of IOP process 1300 in characterizing high amplitude spikes. Additionally, FIG. 16F illustrates the accuracy of simulated ANN pressure calculation with measured pressure.

It should be noted that the algorithms or instructions of IOP measurement process 1300, which includes a signal demodulation, valley/peak detection, spectral features recognition and matching, and an artificial neural network algorithms as previously described, may be stored on a memory that is readable by a computer. A computer may be a processor or an application specific integrated circuit (ASIC). When the algorithms or instructions are executed by the computer, the instructions will cause the computer to carry out the functionalities of IOP measurement process 1300 as described above.

These results suggest that any of IOP sensors 100, 300, 500, 700, 800, 900, and 1000, when processed with the ANN based SDA algorithms, can accurately characterize the intraocular pressure fluctuations ranging from small IOP pulses synchronized with the cardiovascular system to sporadic high amplitude spikes resulting from ocular hypertension or surgical stimulation.

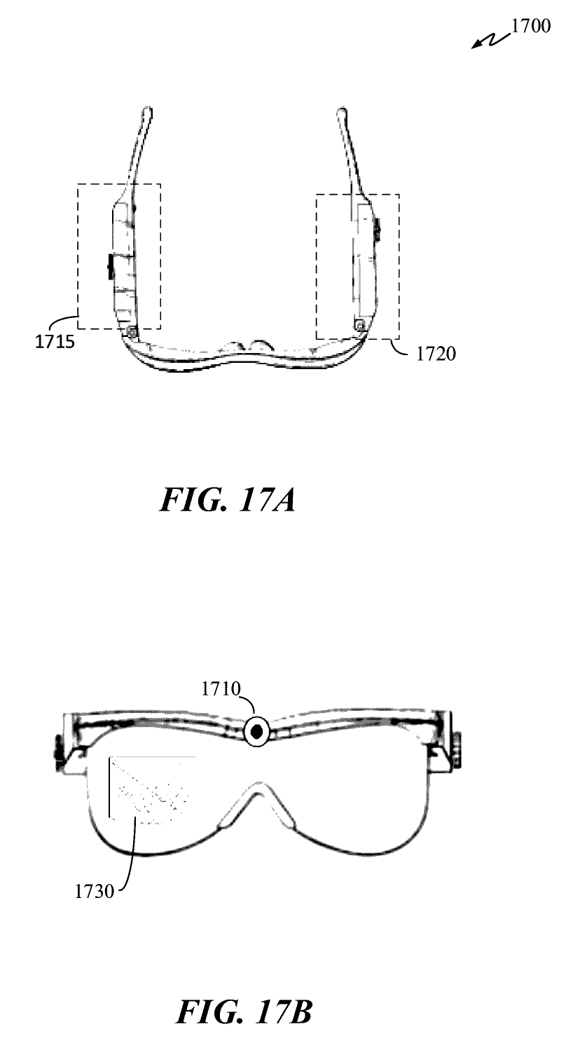

FIGS. 17A-B illustrates a wearable remote optical readout apparatus 1700 in accordance with an aspect of the disclosure. Wearable apparatus 1700 includes an integrated light source and optical sensor 1710. In some embodiments, probe-sensor 1710 may be a commercial 6-around-1 specular reflectance probe having fiber bundle in conjunction with a collimating lens to provide both illumination and detection capabilities. Probe-sensor 1710 may be mounted near the center of wearable apparatus 1700. Alternatively, probe-sensor 1710 may be mounted to the left or right side near one of the ear-mounting frame.

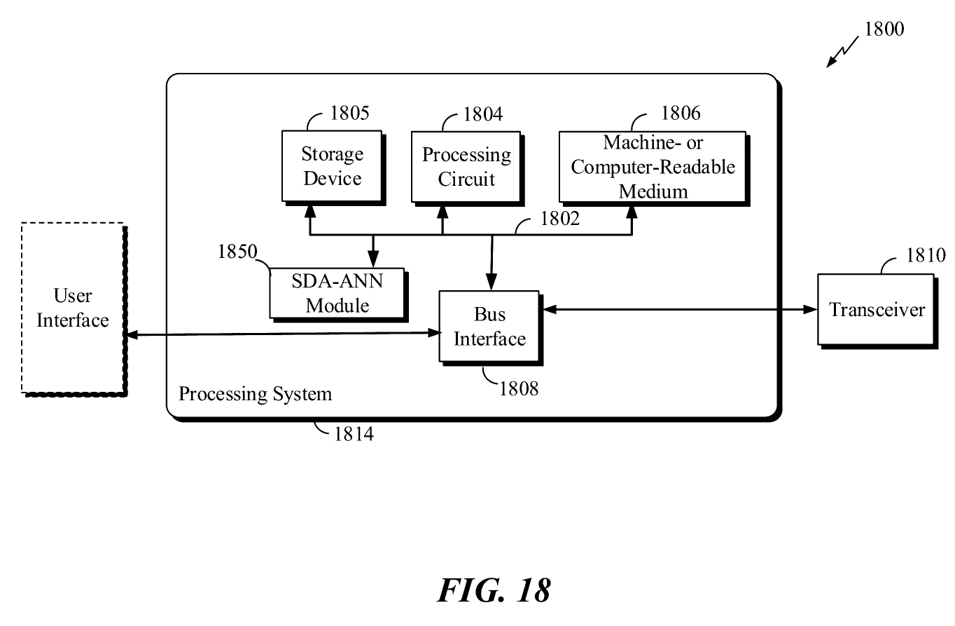

In some embodiments, a broadband light source in the visible or the invisible spectrum can be used. Probe-sensor 1710 can be configured to illuminate the implanted sensor using broadband light and then detect the reflection from the sensor. For IOP readout, the reflected light may be relayed to a commercial mini spectrometer embedded in portion 1715 of apparatus 1700. In some embodiments, wearable apparatus also includes a display 1730 for displaying the IOP readout and/or the resonance spectra of the reflectance light. In some embodiments, wearable apparatus includes an ASIC (see FIG. 18) that is configured to execute IOP process 1300, which includes the SD and ANN algorithms for denoising, peak/valley detection module, reflectance spectra modeling & matching module, and computing the IOP based on the resonance spectra peak/valley profile.

### Example Software Module/Engine and Hardware Implementation

FIG. 18 illustrates an overall system or apparatus 1800 configured to execute IOP measurement process 1300. In accordance with various aspects of the disclosure, an element, or any portion of an element, or any combination of elements may be implemented with a processing system 1814 that includes one or more processing circuits 1804. Processing circuits 1804 may include micro-processing circuits, microcontrollers, digital signal processing circuits (DSPs), field programmable gate arrays (FPGAs), programmable logic devices (PLDs), state machines, gated logic, discrete hardware circuits, and other suitable hardware configured to perform the various functionality described throughout this disclosure. That is, the processing circuit 1804 may be used to implement process 1300 described above and illustrated in FIGS. 13 through 16.

In the example of FIG. 18, the processing system 1814 may be implemented with a bus architecture, represented generally by the bus 1802. The bus 1802 may include any number of interconnecting buses and bridges depending on the specific application of the processing system 1814 and the overall design constraints. The bus 1802 links various circuits including one or more processing circuits (represented generally by the processing circuit 1804), the storage device 1805, and a machine-readable, processor-readable, processing circuit-readable or computer-readable media (represented generally by a non-transitory machine-readable medium 1806.) The bus 1802 may also link various other circuits such as timing sources, peripherals, voltage regulators, and power management circuits, which are well known in the art, and therefore, will not be described any further. The bus interface 1808 provides an interface between bus 1802 and a transceiver 1810. The transceiver 1810 provides a means for communicating with various other apparatus over a transmission medium. Depending upon the nature of the apparatus, a user interface 1812 (e.g., display 1730) may also be provided.

The processing circuit 1804 can be responsible for managing the bus 1802 and for general processing, including the execution of software module/engine stored on the machine-readable medium 1806. In some embodiments, SDA-ANN module 1850 include algorithms as described in process 1300, when executed by processing circuit 1804, causes processing system 1814 to perform the various functions described herein for any particular apparatus. Machine-readable medium 1806 may also be used for storing data that is manipulated by processing circuit 1804 when executing software module/engine.

One or more processing circuits 1804 in the processing system may execute software module/engine or software module/engine components. Software module/engine shall be construed broadly to mean instructions, instruction sets, code, code segments, program code, programs, subprograms, software module/engines/engines, applications, software module/engine applications, software module/engine packages, routines, subroutines, objects, executables, threads of execution, procedures, functions, etc., whether referred to as software module/engine, firmware, middleware, microcode, hardware description language, or otherwise. One or more processing circuits (or processing circuitry) may perform the tasks. A code segment may represent a procedure, a function, a subprogram, a program, a routine, a subroutine, an engine, a module, a software module/engine package, a class, or any combination of instructions, data structures, or program statements. A code segment may be coupled to another code segment or a hardware circuit by passing and/or receiving information, data, arguments, parameters, or memory or storage content. Information, arguments, parameters, data, etc. may be passed, forwarded, or transmitted via any suitable means including memory sharing, message passing, token passing, network transmission, etc.

The software module/engine may reside on machine-readable medium 1806. The machine-readable medium 1806 may be a non-transitory machine-readable medium. A non-transitory processing circuit-readable, machine-readable or computer-readable medium includes, by way of example, a magnetic storage device (e.g., hard disk, solid-state drive), an optical disk (e.g., a compact disc (CD) or a digital versatile disc (DVD)), a smart card, a flash memory device (e.g., a card, a stick, or a key drive), RAM, ROM, a programmable ROM (PROM), an erasable PROM (EPROM), an electrically erasable PROM (EEPROM), a register, a removable disk, a hard disk, a CD-ROM and any other suitable medium for storing software module/engine and/or instructions that may be accessed and read by a machine or computer. The terms “machine-readable medium”, “computer-readable medium”, “processing circuit-readable medium” and/or “processor-readable medium” may include, but are not limited to, non-transitory media such as portable or fixed storage devices, optical storage devices, and various other media capable of storing, containing or carrying instruction(s) and/or data. Thus, the various methods described herein may be fully or partially implemented by instructions and/or data that may be stored in a “machine-readable medium,” “computer-readable medium,” “processing circuit-readable medium” and/or “processor-readable medium” and executed by one or more processing circuits, machines and/or devices. The machine-readable medium may also include, by way of example, a carrier wave, a transmission line, and any other suitable medium for transmitting software module/engine and/or instructions that may be accessed and read by a computer.

The machine-readable medium 1806 may reside in the processing system 1814, external to the processing system 1814, or distributed across multiple entities including the processing system 1814. The machine-readable medium 1806 may be embodied in a computer program product. By way of example, a computer program product may include a machine-readable medium in packaging materials. Those skilled in the art will recognize how best to implement the described functionality presented throughout this disclosure depending on the particular application and the overall design constraints imposed on the overall system.

One or more of the components, steps, features, and/or functions illustrated in the figures may be rearranged and/or combined into a single component, block, feature or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software module/engine and/or embedded in hardware.

Note that the aspects of the present disclosure may be described herein as a process that is depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a function, a procedure, a subroutine, a subprogram, etc. When a process corresponds to a function, its termination corresponds to a return of the function to the calling function or the main function.

While certain example embodiments have been described and shown in the accompanying drawings, it is to be understood that such embodiments are merely illustrative of and not restrictive on the broad invention, and that this invention should not be limited to the specific constructions and arrangements shown and described, since various other modifications are possible. Those skilled in the art will appreciate that various adaptations and modifications of the just described preferred embodiment can be configured without departing from the scope and spirit of the invention. Therefore, it is to be understood that, within the scope of the appended claims, the invention may be practiced other than as specifically described herein.

1. An intraocular pressure sensor comprising:
a first wall comprising a flexible membrane;
a first chamber formed by the first wall and a second wall; and
a first array of photonic components disposed inside of the first chamber.

2. The intraocular pressure sensor of claim 1, further comprising:
a raised portion located within the first chamber on a surface of the second wall, wherein the first array of photonic components is disposed on a surface of the raised portion.

3. The intraocular pressure sensor of claim 2, further comprising a second array of photonic components disposed within the flexible membrane.

4. The intraocular pressure sensor of claim 2, further comprising a second array of photonic components disposed on a surface of the flexible membrane.

5. The intraocular pressure sensor of claim 2, further comprising an optical cavity defined by an air gap between the first array of photonic components and the flexible membrane.

6. The intraocular pressure sensor of claim 1, wherein the first array of photonic components is disposed on the surface of the second wall.

7. The intraocular pressure sensor of claim 6, further comprising a second array of photonic components disposed on a surface of the flexible membrane.

8. The intraocular pressure sensor of claim 7, further comprising an air gap between the first and the second arrays of photonic components.

9. The intraocular pressure sensor of claim 8, wherein the air gap has a thickness range of 5-10 μm.

10. The intraocular pressure sensor of claim 1, wherein the first array of photonic components is disposed on a surface of the flexible membrane.

11. The intraocular pressure sensor of claim 1, wherein the first array of photonic components is located within the flexible membrane.

12. The intraocular pressure sensor of claim 11, further comprising a second array of photonic components disposed on the surface of the second wall.

13. The intraocular pressure sensor of claim 1, wherein the first array of photonic components comprises photonic components with a hemispherical shape.

14. The intraocular pressure sensor of claim 13, wherein each photonic component has a diameter between 200-300 nanometers.

15. The intraocular pressure sensor of claim 13, wherein a distance between a center of a photonic component to a center of an adjacent photonic component in the array of the photonic components is between 250-350 nanometers.

16. The intraocular pressure sensor of claim 1, wherein the first array of photonic components includes photonic components comprising gold.

17. The intraocular pressure sensor of claim 1, wherein the flexible membrane comprises a silicon-nitride based membrane.

18. The intraocular pressure sensor of claim 1, further comprising:
a second chamber sharing the second wall with the first chamber; and
a plurality of openings located on a surface of the second wall to create a plurality of pass-through openings between the first and second chambers, wherein the first array of photonic components is centrally disposed on the surface of the second wall.

19. The intraocular pressure sensor of claim 18, wherein the plurality of openings comprise four openings.

20. The intraocular pressure sensor of claim 18, wherein the second chamber is larger than the first chamber.

21. The intraocular pressure sensor of claim 1, wherein the first array of photonic components comprises a 50 μm×50 μm array of photonic components.

22-50. (canceled)
